SEQUENCE LISTING (4 sequences)

SEQ ID NO: 1 - DNA, 23 bp, Artificial Sequence
  synthetic polynucleotide
atggggggggatggggatggatg

SEQ ID NO: 2 - DNA, 23 bp, artificial sequence
  synthetic polynucleotide
tacccccccctacccctacctac

SEQ ID NO: 3 - DNA, 23 bp, artificial sequence
  synthetic polynucleotide
ttggggggggtaggggatggttg

SEQ ID NO: 4 - DNA, 23 bp, artificial sequence
  synthetic polynucleotide
aaccccccccatcccctaccaac

(19) United States
(12) Patent Application Publication
(10) Pub. No.: US 2014/0234848 A1
(43) Pub. Date: Aug. 21, 2014

(54) Cryptographic Methods Using Nucleic Acid Codes

(71) Applicant: CALIFORNIA INSTITUTE OF TECHNOLOGY, PASADENA, CA (US)

(72) Inventors: Aditya RAJAGOPAL, Irvine, CA (US); Thomas A. TOMBRELLO, Altadena, CA (US)

(73) Assignee: CALIFORNIA INSTITUTE OF TECHNOLOGY, PASADENA, CA (US)

(21) Appl. No.: 14/139,808

(22) Filed: Dec. 23, 2013

Related U.S. Application Data
(60) Provisional application No. 61/756,349, filed on Jan. 24, 2013.

(51) Int. Cl. G06F 21/60; C12Q 1/68

(52) U.S. Cl. CPC G06F 21/602; C12Q 1/686
USPC 435/6.12; 380/255

(57) ABSTRACT

Novel methods and systems for encoding cryptographic information are disclosed. A message can be encoded through a sequence of nucleic acids by assigning a binary value to a pair of nucleic acids, while other nucleic acids can be used for spacing. Unique organisms can also be used for identification. The nucleic acids can be encapsulated in organic materials such as saccharide-based desiccants.

### CROSS REFERENCE TO RELATED APPLICATIONS

The present application claims priority to U.S. Provisional Patent Application No. 61/756,349, filed on Jan. 24, 2013, the disclosure of which is incorporated herein by reference in its entirety.

### TECHNICAL FIELD

The present disclosure relates to cryptography. More particularly, it relates to cryptographic methods using nucleic acid codes.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 illustrates an exemplary method to decode a message from DNA.

FIG. 2 illustrates an exemplary method to transmit a message with DNA.

### SUMMARY

In a first aspect of the disclosure, a method to encode cryptographic information is described, the method comprising: providing a message to be encoded; defining a truth table, the truth table determining a correspondence between sequences of nucleic acids and a code; providing nucleic acids; and arranging said nucleic acids in a sequence, according to the truth table and the message, thereby encoding the message in the sequence of said nucleic acids.

In a second aspect of the disclosure, a method to encode cryptographic information is described, the method comprising: providing a number of unique organisms; defining a truth table, the truth table determining a correspondence between the number of unique organisms and a code; and selecting a group of the unique organisms, according to the truth table and the message, thereby encoding the message in the group of unique organisms.

In a third aspect of the disclosure, a method to encode cryptographic information is described, the method comprising: providing benign organisms; inserting the benign organisms in a human or animal; and identifying the human or animal through the benign organisms.

In a fourth aspect of the disclosure, a method to encode cryptographic information is described, the method comprising: transfecting a host with a nucleic acid target, the nucleic acid target coding a unique protein; and identifying the host by processing an immunoassay on a blood sample of the host containing the unique protein.

### DETAILED DESCRIPTION

Cryptographic techniques are used for the encoding and decoding of information that needs to be hidden from all possible recipients except for the intended target. However, efficient codes must strike a balance between complexity of coding and density of information. Furthermore, the interpretation of these codes should be done in such a way that a reader (intended recipient) can decode the information in an unambiguous fashion. Elegant codes can achieve this with one-to-one mapping of information to a cryptogram. For instance, a one-to-one mapping of a message in English would require 26 differently coded characters. Effectively, this requires a base-26 code for the mapping.

“Binary code” as used herein, refers to a text or computer processor instructions that use a binary number system's two binary digits, 0 and 1. A binary code assigns a bit string to each symbol or instruction. For example, a binary string of eight binary digits (bits) can represent any of 256 possible values and can therefore correspond to a variety of different symbols, letters or instructions.

Binary codes can be used for various methods of encoding data, such as character strings, into bit strings. Those methods may use fixed-width or variable-width strings. In a fixed-width binary code, each letter, digit, or other character is represented by a bit string of the same length; that bit string, interpreted as a binary number, is usually displayed in code tables in octal, decimal or hexadecimal notation. There are many character sets and many character encodings for them.

There are other ways of replicating this type of code. Binary values of a predetermined length can be used to code for letters and symbols. For instance, the English language ASCII lookup table maps a hexadecimal (effectively binary) code to every character of the language. This allows for the coding of letters in a fashion that can be understood by machines.

As described in the present disclosure, it is also possible to encode messages using nucleic acids, which is synonymous with polynucleotides. As used herein, “nucleic acids” are linear polymers (chains) of nucleotides. Each nucleotide consists of three components: a purine or pyrimidine nucleobase (sometimes termed nitrogenous base or simply base), a pentose sugar, and a phosphate group. The substructure consisting of a nucleobase plus sugar is termed a nucleoside. Nucleic acid types differ in the structure of the sugar in their nucleotides—dideoxyribonucleic acid (DNA) contains 2′-deoxyribose while ribonucleic acid (RNA) contains ribose (where the only difference is the presence of a hydroxyl group). Also, the nucleobases found in the two nucleic acid types are different: adenine, cytosine, and guanine are found in both RNA and DNA, while thymine occurs in DNA and uracil occurs in RNA. In the following, as the person skilled in the art will understand, A stands for adenine, C for cytosine, G for guanine, U stands for uracil, and T for thymine.

In one embodiment, a scheme can be used, wherein the A-T 2-mers code for the digit 0, while A-A and T-T 2-mers code for the digit 1. In this case, a reading of either A-T or T-A is interpreted as a 0, while the reading of either A-A or T-T is interpreted as a 1.

Since DNA is readily stabilized by its complementary strand, this allows for a natural redundancy in the message contents. In this coding scheme, the reading frame is determined by the binary spacing of the intervening G's and C's. For instance, the following sequence can be considered:

(SEQ ID NO: l)
5′ATGGGGGGGGATGGGGATGGATG3′
(SEQ ID NO: 2)
3′TACCCCCCCCTACCCCTACCTAC5′

As the person skilled in the art will understand, in the sequence above the nucleic acid pairs are lined up. In the sequence above, the message is read starting at the 5′ end of the bottom strand. This is due to the fact that the G-C spacing increases as the frame shifts downstream (to the 3′ end). The coding base pairs (A's and T's) can be tagged with a variety of reporters including but not limited to heavy metal ionic tags, fluorescent markers, quantum dots, quenchers, hydronium (ph-based) markers, and radioactive tags. The message contents are read by sequencing the entire DNA strand. The spacing between the reporters allows the reader to determine the location of the message that is being read, while the tags themselves denote how the content is to be interpreted.

Another sequence can be considered, carrying two bit information:

(SEQ ID NO: 3)
5′TTGGGGGGGGTAGGGGATGGTTG3′
(SEQ ID NO: 4)
3′AACCCCCCCCATCCCCTACCAAC3′

The information in the sequence above can be interpreted, for example, through the truth table for two-bit binary coding in Table 1.

TABLE 1

| Code | Interpretation |
|---|---|
| 5′ AT3′ | 0 |
| 5′ TA3′ | 0 |
| 5′ AA3′ | 1 |
| 5′ TT3′ | 1 |

An example of a two-bit base-four truth table can be found in Table 2.

TABLE 2

| Code | Interpretation |
|---|---|
| 5′ AT3′ | 0 |
| 5′ TA3′ | 1 |
| 5′ AA3′ | 3 |
| 5′ TT3′ | 4 |

In the examples above, the actual information is carried by the A-T pairs, while G and C act as spacing.

Nucleic acids carrying an encoded message can be encapsulated in organic materials.

Nucleic acid cryptograms can be easily transported in a variety of organic desiccants that preserve the bond structures while preventing contamination from external environment. One such embodiment is the inclusion of nucleic acid cryptograms in a saccharide-based desiccant. For instance, the cryptograms can be preserved inside of sucrose packages for transportation. In addition to preserving the codes, the delivery method allows for easy destruction of the message if the need should arise. The message contents can be easily and safely digested by humans or animals. Furthermore, the content of the message can be obfuscated, destroyed, and disposed of by dissolving the saccharide package in a solution including but not limited to saliva, water, urine, soda, ethanol, and alcoholic beverages.

The form factor for this packaging embodiment is particularly advantageous, since it is very similar to hard candy. This allows for the package to be physically transported from the creator to the intended recipient without arousing suspicion.

When the content of the message needs to be read, the saccharide package can be dissolved in a known solution that is conducive to nucleic acid stability. An electric field can be applied to extract and concentrate the nucleic acid cryptogram. Simple sequencing can be used to determine the content of the message.

In other embodiments, nucleic acid coding can involve the use of n unique organisms (such as bacteriophages, bacteria, or other viruses). Each organism represents a “bit” of the message. For instance, if organisms 1, 3, 6, and 19 are present, in a 20-bit protocol, the code could be read as: 01000 00000 00001 00101. In other words, by having 20 different organisms, each organism can encode a code of a 20-bit protocol. In this case n=20, but different numbers may be used.

In different embodiments different coding schemes can be used. In order to decode the message, the recipient would only need to run a multiplexed PCR reaction containing the message. This allows for fast decoding of the content of the message.

Several techniques could be used for the transportation of the message. Providing a natural environment for each “bit” of the message would likely preserve the content for an appreciable amount of time. For instance, in an n-bit code each organism can be stored in a host such as a small rodent or mammal. In order to decode the message, the recipient would only need to draw the blood of the animal and run a PCR reaction. The message host provides the ideal conditions to preserve the message, while additionally degrading the contents of the message for prolonged time scales (i.e. the host will die due to infection).

The term “Single nucleotide polymorphisms” or “SNPs” as used herein, refers to a DNA sequence variation occurring when a single nucleotide—A, T, C or G—in the genome (or other shared sequence) differs between members of a biological species or paired chromosomes in a human. For example, two sequenced DNA fragments from different individuals, AAGCCTA to AAGCTTA, contain a difference in a single nucleotide. In this case we say that there are two alleles, which are one of a number of alternative forms of the same gene or same genetic locus. Almost all common SNPs have only two alleles. The genomic distribution of SNPs is not homogenous; SNPs can occur in non-coding regions more frequently than in coding regions or, in general, where natural selection is acting and fixating the allele of the SNP that constitutes the most favorable genetic adaptation. Other factors, like genetic recombination and mutation rate, can also determine SNP density. SNP density can be predicted by the presence of microsatellites, which are repeating sequences of 2-6 base pairs of DNA: AT microsatellites in particular are potent predictors of SNP density, with long (AT)(n) repeat tracts tending to be found in regions of significantly reduced SNP density and low GC content. Within a population, SNPs can be assigned a minor allele frequency—the lowest allele frequency at a locus that is observed in a particular population. This is simply the lesser of the two allele frequencies for single-nucleotide polymorphisms. There are variations between human populations, so a SNP allele that is common in one geographical or ethnic group may be much rarer in another. These genetic variations between individuals (particularly in non-coding parts of the genome) can be exploited in DNA fingerprinting, which is used in forensic science. In an embodiment described herein, the genetic variation, or SNPs can be used as an identifier or a secondary message within the encrypted message, which uses the SNP as an authentication code for the specific organism or carrier of the encrypted message.

The term “genotype” as used herein, refers to the genetic makeup of a cell, an organism, or an individual usually with reference to a specific characteristic under consideration. For example a specific organism can have a SNP genotype associated with the organism. In some embodiments described herein, the message can be placed within a gene that has a specific SNPs that can be used in a search to provide authentication of the encrypted message.

Finally, hosts can be intentionally tagged with benign organisms for identification purposes. For instance, a predetermined set of non lethal bacteria or viruses can be used to infect a human host. When the human host is required to provide authentication of their identity, a drop of blood can be extracted from them. The PCR analysis run on the blood would confirm their identity. Because of the specificity of the PCR reaction, only the intended authenticator would know how to interpret the nucleic acid contents of the blood sample. Furthermore, this has the additional benefit of obfuscating the contents of the message (i.e. blood sample), since the blood will contain native host DNA as well as the DNA of any parasitic and symbiotic organisms.

In other embodiments, organisms can be used to transfect the host with particular nucleic acid targets. These targets would code for unique proteins that would be expressed in the blood. In order to read the message contents, the blood contents would only need to be processed in an immunoassay such as ELISA. The specificity of the antibody-protein bond would allow the message to be uniquely interpreted.

In another embodiment a competent cell bacteria for high replication of DNA plasmid can be transformed with a vector or plasmid, preferably a PET vector for high replication in bacteria, containing the DNA message within two restriction sites. The bacteria can be sent to a recipient in a slab sample. In order to decode the message, the recipient can then grow up the bacteria in a Luria Broth culture, and perform a DNA extraction preparation such as a mini-prep, or a maxi-prep according to the Qiagen methods, which is known to those skilled in the art. The DNA that is purified can then be amplified by PCR techniques using primers at the 3′ and 5′ end of the DNA message that are specific for the DNA restriction sites which flank the DNA message. The samples can be amplified using a standard PCR machinery such as GeneAmp® PCR System 9700, and the PCR product which contains the message can then be purified using a Qiagen PCR purification kit, and analyzed using standard agarose gel techniques to examine the DNA message sizes. The PCR product along with the amplifying primers can then be sequenced. For example, the PCR product along with the amplifying primers can be sent to a sequencing company such as Integrated DNA Technologies, which can send zip files of the DNA sequences which can then be translated through computation methods by the recipient.

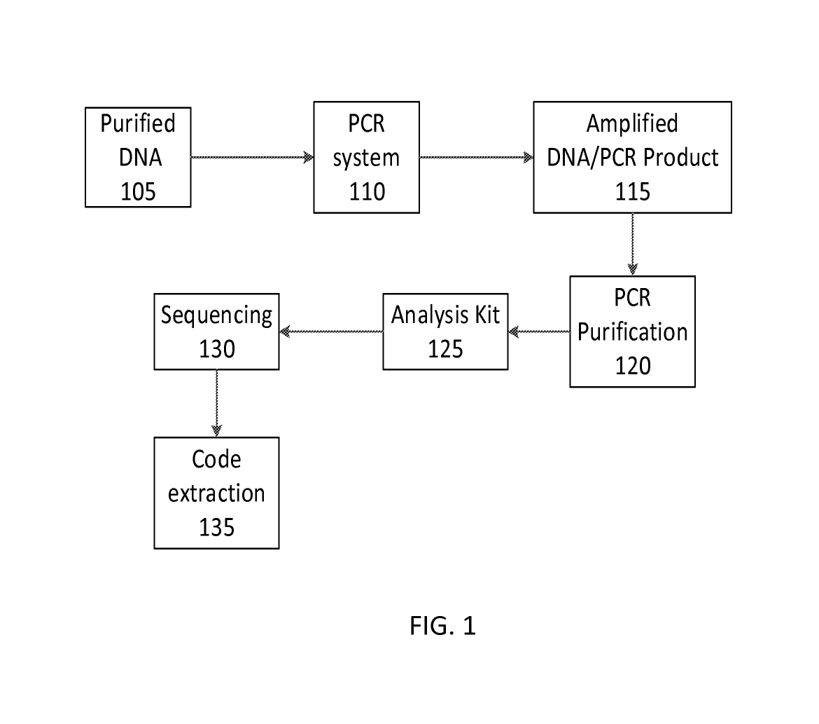

FIG. 1 illustrates an exemplary method to decode a message from DNA. The purified DNA (105) is processed by a PCR system (110). The resultant amplified DNA or PCR product (115) is subsequently processed with a PCR purification kit (120) and then analyzed (125). The resultant DNA can be sequenced (130) and then the message can be decoded from the DNA (135).

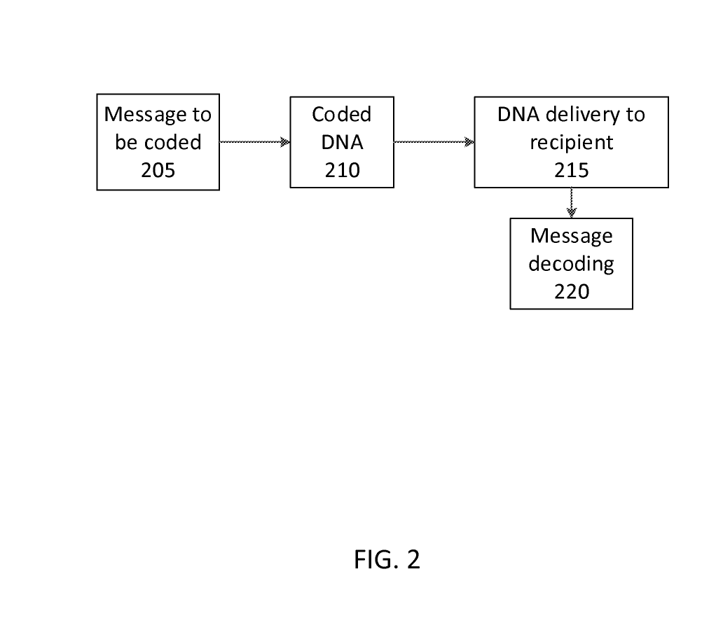

FIG. 2 illustrates an exemplary method to encode a message in DNA. The message to be encoded (205) is coded in DNA (210), which is then delivered to the recipient (215). The message can then be decoded from the DNA (220).

An example of an organism-based 20-bit encoding can be found in Table 3.

TABLE 3

| Code | Interpretation |
|---|---|
| 1, 2, 3 | 00000 00000 00000 00111 |
| 1, 2, 5 | 00000 00000 00000 10011 |
| 2, 6, 12 | 00000 00010 00001 00010 |
| 1, 3, 6, 19 | 01000 00000 00001 00101 |

In some embodiments, the sequence of nucleic acids is belongs to an animal, a plant or organic products.

In some embodiments, the sequence of bases comprises not only natural base pairs such as A, C, G, and T/U, but also unnatural or artificial base pairs. In some embodiments, the sequence of bases comprises only artificial base pairs.

In some embodiments, the nuclei acid can store a coded message, while other groups of nucleic acids can store a cryptographic key to a coded message.

In some embodiments, G & C can be used for encoding information and A & T can be used for spacing. There may be a relative advantage to using this over the opposite scheme, in certain scenarios, since the G-C bond has a higher bond energy (and hence is more stable) than the A-T bond.

A number of embodiments of the disclosure have been described. Nevertheless, it will be understood that various modifications may be made without departing from the spirit and scope of the present disclosure. Accordingly, other embodiments are within the scope of the following claims.

The examples set forth above are provided to those of ordinary skill in the art as a complete disclosure and description of how to make and use the embodiments of the disclosure, and are not intended to limit the scope of what the inventor/inventors regard as their disclosure.

Modifications of the above-described modes for carrying out the methods and systems herein disclosed that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

It is to be understood that the disclosure is not limited to particular methods or systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence. Further, the paper copy of the sequence listing submitted herewith and the corresponding computer readable form are both incorporated herein by reference in their entireties.

What is claimed is:

1. A method to encode cryptographic information, the method comprising:
providing a message to be encoded;
defining a truth table, the truth table determining a correspondence between sequences of nucleic acids and a code;
providing nucleic acids; and
arranging said nucleic acids in a sequence, according to the truth table and the message, thereby encoding the message in the sequence of said nucleic acids.

2. The method of claim 1, wherein the defining the truth table comprises encoding information using adenine and thyamine.

3. The method of claim 1, wherein the defining the truth table comprises assigning adenyne-thyamine and thyamine-adenine to 0, and adenine-adenine and thyamine-thyamine to 1.

4. The method of claim 2, wherein the arranging comprises using guanine and cytosine as spacers.

5. The method of claim 1, further comprising encapsulating the sequence of nucleic acids in an organic material.

6. The method of claim 5, wherein the organic material is a saccharide-based desiccant.

7. The method of claim 6, further comprising:
dissolving the organic material;
applying an electric field to extract and concentrate the sequence of nucleic acids; and
sequencing the nucleic acids.

8. The method of claim 1, further comprising tagging the sequence of nucleic acids with one or more of: heavy metal ionic tags, fluorescent markers, quantum dots, quenchers, hydronium (ph-based) markers, or radioactive tags.

9. The method of claim 1, wherein the defining the truth table comprises encoding information using guanine and cytosine, and wherein the arranging comprises using adenine and thyamine as spacers.

10. The method of claim 9, wherein the defining the truth table further comprises encoding single nucleotide polymorphisms (SNPs) for a genotype.

11. A method to encode cryptographic information, the method comprising:
providing a number of unique organisms;
defining a truth table, the truth table determining a correspondence between the number of unique organisms and a code; and
selecting a group of the unique organisms, according to the truth table and the message, thereby encoding the message in the group of unique organisms.

12. The method of claim 11, wherein the unique organisms are one or more of: bacteriophages, bacteria, or viruses.

13. The method of claim 11, further comprising hosting the group of unique organisms in a human or animal.

14. The method of claim 1, further comprising hosting the sequence of nucleic acids in a human or animal.

15. The method of claim 1, wherein the sequence of nucleic acids is of an animal, a plant or organic products.

16. The method of claim 1, further comprising encoding a cryptographic key separately from the message.

17. The method of claim 1, wherein the nucleic acids are in artificial pairs.

18. The method of claim 15, wherein the sequence of nucleic acids of an animal or a plant comprises specific single nucleotide polymorphisms (SNPs) for a specific genotype of the animal or of the plant.

19. A method to encode cryptographic information, the method comprising:
providing benign organisms;
inserting the benign organisms in a human or animal; and
identifying the human or animal through the benign organisms.

20. The method of claim 18, wherein the benign organisms are non lethal bacteria or viruses.

21. A method to encode cryptographic information, the method comprising:
transfecting a host with a nucleic acid target, the nucleic acid target coding a unique protein; and
identifying the host by processing an immunoassay on a blood sample of the host containing the unique protein.
